(19) United States
(12) Patent Application Publication
(10) Pub. No.: US 2026/0226383 A1
(43) Pub. Date: Aug. 6, 2026

(54) DIGITAL MOLECULAR FINGERPRINTING ON ORGAN ON A CHIP SYSTEMS

(71) Applicants: UNIVERSITY OF ROCHESTER, Rochester, NY (US); THE REGENTS OF THE UNIVERSITY OF MICHIGAN, Ann Arbor, MI (US)

(72) Inventors: James L. MCGRATH, Rochester, MI (US); Benjamin H. SINGER, Ann Arbor, MI (US); Andrew STEPHENS, Ann Arbor, MI (US); Yujing SONG, Ann Arbor, MI (US); Katsuo KURABAYASHI, Ann Arbor, MI (US); Shiuan-Haur SU, Ann Arbor, MI (US)

(21) Appl. No.: 19/142,420

(22) PCT Filed: Dec. 13, 2023
(86) PCT No.: PCT/US2023/083877

§ 371 Date: Jun. 23, 2025

Related U.S. Application Data
(60) Provisional application No. 63/477,130, filed on Dec. 23, 2022.

(51) Int. Cl. C12M 3/06; C12M 1/00; G01N 33/50; G01N 33/543

(52) U.S. Cl. CPC C12M 23/16; C12M 29/04; G01N 33/5058; G01N 33/54366

(57) ABSTRACT

The present disclosure relates to systems, devices, and methods for using a first component (e.g., having a top reservoir and bottom reservoir that are fluidically connected through, and only through, a nanoporous membrane) that is able to be mated with a second component that comprises a plurality of microwells. Stimulating agents and/or candidate therapeutics may be added to the top reservoir such that cells (e.g., endothelial cells) in the top reservoir release one or more analytes that pass through the nanoporous membrane into the bottom reservoir (which may contain cells such as neurons) and into the microwells where they are captured and then detected.

The present application present application claims priority to U.S. Provisional application Ser. No. 63/477,130 filed Dec. 23, 2022, which is herein incorporated by reference in its entirety.

This invention was made with government support under HL154249, AG074968, and NS101054 awarded by the National Institutes of Health, and under CBET1931905 awarded by the National Science Foundation. The government has certain rights in the invention.

### FIELD OF THE INVENTION

The present disclosure relates to systems, devices, and methods for using a first component (e.g., having a top reservoir and bottom reservoir that are fluidically connected through, and only through, a nanoporous membrane) that is able to be mated with a second component that comprises a plurality of microwells. Stimulating agents and/or candidate therapeutics may be added to the top reservoir such that cells (e.g., endothelial cells) in the top reservoir release one or more analytes that pass through the nanoporous membrane into the bottom reservoir (which may contain cells such as neurons) and into the microwells where they are captured and then detected.

### BACKGROUND OF THE INVENTION

Recent advances in organ-on-a-chip (OoC) technology have enabled researchers to create engineered or natural tissues within microfluidic chips. Much promise arises in observing the tissues' physiological phenomena under OoC conditions replicating complex in vivo biological environments (Low et al. 2021). Animal models are the current gold standard for preclinical validation of therapeutics. However, animal studies require breeding, dissections, and ethical clearances. Preclinical studies in the U.S. use as many as 111 million mice every year (Carbone 2021), which is a huge cost to the biomedical research and drug discovery industry. More importantly, cross-species differences between animals and humans have failed many animal studies attempting to develop a therapy for the human central nervous system (CNS). Replacing animal models with OoC platforms can eliminate these issues and provide accessibility and throughput for blood brain barrier (BBB) studies.

The BBB is a highly selective vascular border of the CNS with complex functions, including transporting metabolic products, preventing crossing of bloodborne pathogens and neurotoxic substances, and regulating immune responses on the fragile abluminal side (Obermeier et al. 2013). One of the OoC subsets, a BBB-on-a-chip (BBB-oC), facilitates BBB-penetrable drug discovery or studies of CNS disease mechanisms. Researchers have studied the integrity of the barrier during BBB-oC experiments with integrated sensors (Ahn et al. 2020; Badiola-Mateos et al. 2021; Yu et al. 2020). However, these studies have been limited to using trans-endothelial electrical resistance (TEER) sensors to characterize membrane permeability and integrity (Liang and Yoon 2021; Mir et al. 2022; Wolff et al. 2015). The electrophysical measurements only provide a nonspecific signature of the membrane functionality, masking multiple combined biological factors.

Immunosensors previously integrated into other OoC systems include localized surface plasmon resonance (LSPR) sensors for adipose tissue (Zhu et al. 2018) and electrochemical sensors for heart, liver organoids and muscle tissues (Ortega et al. 2019; Zhang et al. 2017). However, the poor linear range of these sensors (Zhang and Noji 2017) limits their direct implementation in BBB-oC immunosensing. An ideal BBB-oC experiment generally necessitates a high throughput platform integrated with multiplexed, sensitive immunosensors having large dynamic ranges. The BBB-oC allows one to measure low-abundance neurotoxicity-inducing biomarkers, such as cytokines, and monitor multiple time-varying protein secretion behaviors of the BBB model. The synthesis and release of cytokines are highly regulated and temporally orchestrated in the immune system (Lacy and Stow 2011). Through positive feedback, inflammatory cytokines induce a self-perpetuating cascade that synergistically upregulates other inflammation biomarkers like chemokines and cell adhesion molecules (CAMs) (Lecuyer et al. 2016; Lombardi et al. 2009). However, the mere presence of some inflammatory chemokines like MCP1 may not indicate neuroinflammation but offer neuroprotective functions to the BBB (Schilling et al. 2009; Stowe et al. 2012; Williams et al. 2014). Thus, temporal monitoring of the homeostatic BBB cytokine levels, immune cascade onset, progression, and resolution can elucidate critical information such as the “tipping point” to sustained chronic neuroinflammation and the true physiological picture (DiSabato et al. 2016; Gilroy and Lawrence 2008). This information can be obtained through multiple endpoint measurements in some situations. However, these measurements waste time, materials, and sample resources and introduce additional batch effects that decrease reproducibility. Achieving a high throughput assay can suppress cellular batch effects and mitigate the long sample preparation time usually accompanying BBB cell culture preparation and induced pluripotent stem cells (iPSC)-related applications. Unfortunately, conventional immunosensors fail to meet these needs as they cannot detect a subtle concentration change of low abundance analytes or achieve multiplex capabilities for a panel of markers co-existing in highly different concentration ranges. As a result, a BBB-oC experiment with these immunosensors cannot trace rare biomarkers or probe the tissue's abrupt, temporally resolved secretomic responses. Furthermore, integrating these immunosensors often requires complex fluidic circuits, making the BBB-oC platform inaccessible to non-engineering labs.

### SUMMARY OF THE INVENTION

The present disclosure relates to systems, devices, and methods for using a first component (e.g., having a top reservoir and bottom reservoir that are fluidically connected through, and only through, a nanoporous membrane) that is able to be mated with a second component that comprises a plurality of microwells. Stimulating agents and/or candidate therapeutics may be added to the top reservoir such that cells (e.g., endothelial cells) in the top reservoir release one or more analytes that pass through the nanoporous membrane into the bottom reservoir (which may contain cells such as neurons) and into the microwells where they are captured and then detected.

In some embodiments, provided herein are systems comprising: a) a first component comprising: i) a housing block having a top surface, a bottom surface, and top reservoir recessed in the top surface, wherein the top reservoir comprises: one or more side walls and a bottom wall in proximity to the bottom surface of the housing block and having a reservoir hole therein, ii) a membrane chip comprising a membrane housing and a nanoporous membrane, wherein the membrane chip is configured to sit on or near the bottom wall of the top reservoir such that the nanoporous membrane protrudes through the reservoir hole out past the bottom surface of the housing block, and iii) optionally an adhesive layer that is sized to stick to the bottom surface of the housing block, and iv) a film layer with a top surface and a bottom surface, wherein the film layer has an opening therein, wherein the opening comprises a film perimeter wall that forms a bottom reservoir when: A) the top surface of the film layer is mated with the bottom surface of the housing block, optionally via the adhesive layer, and B) the bottom surface of the film layer is mated with a second component, optionally via pressure.

In particular embodiments, the top reservoir and the bottom reservoir are fluidically connected through, and only through, the nanoporous membrane when the housing block, membrane chip, and the film are assembled together, optionally with the adhesive layer. In certain embodiments, the housing block, membrane chip, and the film are assembled together, optionally via the adhesive layer. In additional embodiments, the adhesive layer is present in the system and comprises two-sided film adhesive. In some embodiments, the bottom surface of the film is mated with the second component via pressure. In some embodiments of the systems, they further comprise the second component.

In particular embodiments, provided herein are devices comprising: a first component which comprises: a) a housing block having a top surface, a bottom surface, and top reservoir recessed in the top surface, wherein the top reservoir comprises: one or more side walls and a bottom wall in proximity to the bottom surface of the housing block and having a reservoir hole therein, b) a membrane chip comprising a membrane housing and a nanoporous membrane, wherein the membrane chip is seated on or near the bottom wall of the top reservoir such that the nanoporous membrane protrudes through the reservoir hole out past the bottom surface of the housing block, and c) optionally an adhesive layer that is stuck to the bottom surface of the housing block, and d) a film layer with a top surface and a bottom surface, wherein the film layer has an opening therein, wherein the opening comprises a film perimeter wall that forms a bottom reservoir when: A) the top surface of the film layer is mated with the bottom surface of the housing block, optionally via the adhesive layer, and B) the bottom surface of the film layer is mated with a second component, optionally via pressure.

In certain embodiments, the top reservoir and the bottom reservoir are fluidically connected through, and only through, the nanoporous membrane. In particular embodiments, a plurality of the first components are present in the device, and optionally wherein the plurality is 4, 6, or 8 first components. In some embodiments, the second component comprises a microwell chip, wherein the microwell chip comprises a surface with a plurality of microwells therein, and wherein optionally each of the microwells has a diameter of about 3-5 micrometers or about 2-10 micrometers, and optionally wherein each of the microwells has a volume of 1-10 femtoliters or 1-00 femtoliters.

In certain embodiments, provided herein are methods comprising: a) adding a first cell suspension comprising a plurality of cells to the top reservoir of the first component of the devices detailed above and herein, wherein the first component is sealed against a second component such that the bottom reservoir is formed, wherein the second component comprises a microwell chip, wherein the first location on the microwell chip comprises a surface with a first plurality of microwells therein, wherein each of the first plurality of microwells comprises analyte binding agents, and wherein the sealing is such that the plurality of microwells are in fluidic communication with the bottom reservoir, b) adding a stimulating agent and/or candidate therapeutic to the top reservoir of the first component under conditions such that at least some of the plurality of cells secrete one or more analytes that: i) pass through the nanoporous membrane, ii) flow into the bottom reservoir, and iii) flow into at least a portion of the first plurality of microwells; and c) detecting any bound analytes in at least a portion of the first plurality of microwells at a first time.

In certain embodiments, provided herein are methods comprising: a) sealing the film layer of the first component of the devices described above and herein against a first location on a second component such that the bottom reservoir is formed, wherein the second component comprises a microwell chip, wherein the first location on the microwell chip comprises a surface with a first plurality of microwells therein, wherein each of the first plurality of microwells comprises analyte binding agents, and wherein the sealing is such that the plurality of microwells are in fluidic communication with the bottom reservoir, b) adding a first cell suspension comprising a plurality of cells to the top reservoir of the first component, c) adding a stimulating agent and/or candidate therapeutic to the top reservoir of the first component under conditions such that at least some of the plurality of cells secrete one or more analytes that: i) pass through the nanoporous membrane, ii) flow into the bottom reservoir, and iii) flow into at least a portion of the first plurality of microwells; and d) detecting any bound analytes in the a least a portion of the first plurality of microwells at a first time.

In some embodiments, the detecting is performed by optical detection of an added label. In particular embodiments, the methods further comprise: e) moving the first component to a second location on the second component such that the bottom reservoir remains formed, wherein the second location on the microwell chip comprises a surface with a second plurality of microwells therein, wherein the second plurality of microwells comprises analyte binding agents. In other embodiments, the moving comprises sliding the first component across the second component from the first location to the second location. In additional embodiments, further comprising: f) detecting any bound analytes in a least a portion of the second plurality of microwells at a second time. In other embodiments, the detecting is performed by optical detection of an added label.

In some embodiments, the methods further comprise: g) moving the first component to a third location on the second component such that the bottom reservoir remains formed, wherein the second location on the microwell chip comprises a surface with a third plurality of microwells therein, wherein the third plurality of microwells comprises analyte binding agents. In particular embodiments, the moving comprises sliding the first component across the second component from the second location to the third location. In other embodiments, the methods further comprise: h) detecting any bound analytes in at least a portion of the third plurality of microwells at a third time. In additional embodiments, the detecting is performed by optical detection of an added label. In certain embodiments, the top reservoir and the bottom reservoir are fluidically connected through, and only through, the nanoporous membrane.

In some embodiments, provided herein are the use of the systems or devices described above and herein in a method of detecting bound analytes in the plurality of microwells, such as after adding a stimulating agent and/or candidate therapeutic to the top reservoir of the first component.

In other embodiments, the plurality of microwells are in fluidic communication with the bottom reservoir when the first component is assembled and mated with the second component. In additional embodiments, the microwell chip comprises glass. In further embodiments, the plurality of microwells comprises at least 1000, at least 10,000, at least 75,000 microwells, or at least 89,000 microwells. In additional embodiments, the plurality of microwells are present in at least two or at least three distinct arrays. In particular embodiments, each of the arrays comprises at least 1000, at least 10,000, or at least 75,000 microwells, or at least 89,000 microwells.

In certain embodiments, each of the plurality of microwells comprises an analyte binding agent. In some embodiments, the analyte binding agent comprises an antibody or antigen binding fragment thereof, and optionally wherein the antibody or antigen binding fragments thereof and the plurality of microwells form hydrophilic-in-hydrophobic capture-antibody-functioned microwells. In other embodiments, the analyte comprises a cytokine or chemokine. In additional embodiments, each of the at least two or at least three distinct arrays comprises a different analyte binding agent configured to bind a different analyte.

In some embodiments, the top reservoir comprises a first suspension of cells, and optionally a stimulating agent or candidate therapeutic, wherein the stimulating agent is optionally LPS, and wherein the candidate therapeutic is optionally a small molecule, antibody or antigen binding fragment thereof, a nanoparticle, or a liposome. In additional embodiments, the first suspension of cells comprises endothelial cells, which has optionally formed a layer on the nanoporous membrane. In certain embodiments, the endothelial cells comprise brain microvascular endothelial cells. In some embodiments, the first component is assembled and mated with the second component such that the bottom reservoir is formed, and wherein the bottom reservoir comprises a second suspension of cells.

In additional embodiments, the second suspension of cells comprises neurological cells or brain cells. In some embodiments, the neurological cells comprise neurons. In further embodiments, the nanoporous membrane comprise a silicon-nitride membrane, which optionally is transparent for optical imaging. In other embodiments, the housing block comprises plastic, optionally acrylic. In particular embodiments, the film layer is composed of PDMS.

### DESCRIPTION OF THE DRAWINGS

FIG. 1 (A) Schematic of an integrated DigiTACK measurement of abluminal (brain)-side cytokine concentration via spatially-encoded-antibody-functioned microwells. Upon LPS stimulation, the brain microvascular endothelial cells secrete significant amounts of MCP1, IL6 and KC to both sides of the barrier which could cause systemic neuroinflammation. Neurons are drawn with dashed outlines to represent the abluminal side of the barrier system and may, or may not be, present. The captured analytes are later digitally counted for concentration quantification. (B)(i) Exploded view of one exemplary DigiTACK device which contains two components that are reversibly sealed. Component 1: A nanoporous silicon nitride (NPN) membrane chip is mounted onto a recessed acrylic membrane housing block. The bottom surface of the housing block is bonded to a PDMS thin film through-cut channel through a double-sided PSA. Together, Component 1 creates a Transwell-style open well reservoir for luminal (blood) fluid/stimulation in the top compartment and a bottom abluminal microfluidic channel compartment. The two compartments are connected only through the suspended NPN/cell membrane interface. Component 2: A glass chip with arrayed digital sensor patches fabricated through DGRIE and micropatterning. Each digital sensor patch contains 3 rectangular arrays (89,760 microwells/array) that fits under the PDMS abluminal channel with each array targeting a different analyte. The soft bottom PDMS thin film channel layer of Component 1 acts like an O-ring that allows reversible sealing to component 2. (ii) Phase contrast image of brain microvascular endothelial cells cultured in the device. (iii) SEM image of a NPN membrane. The membrane has a ~15% porosity (60 nm pores) and a ~50 nm thickness. (iv) Brightfield image of the DGRIE-etched glass microwells. Microwells are 3.8 μm in diameter and 3.5 μm in depth. (v) Fluorescent image of antibody specifically functioned within the microwells. Alexa fluor 488 anti-rat-IgG antibodies were used to label and visualize the distribution of mouse IL6 capture antibodies.

FIG. 2. Exemplary assay procedure of multiplexed longitudinal fingerprinting of same devices. (A) At the first time point of interest (T1), an arrayed (2×4) Component 1 strip (8 devices with different stimulation conditions or replicates) along with its abluminal fluid is moved and reversibly sealed to a set of digital sensor patches on a Component 2. The exposed patches are in contact with the abluminal fluid for 15 min, which captures and records concentrations of the 3 analytes within all 8 devices at designated positions. (B) At T2, the Component 1 strip and the bottom fluid is moved and resealed to the next set of digital sensor patches on Component 2. This again records the 3-plexed concentration information at T2 across all 8 devices. (C) Similar to (B), the Component 1 strip is moved to the third set of digital sensor patches and the same multiplexed information is captured for T3. 10 μL of supernatants from the top luminal reservoir was also sampled and frozen at each time point for further concentration measurements. (D) The Component 1 strip is replaced with one PDMS channel (PDMS assay layer) on top of the Component 2 to perform high throughput detection antibody labeling, HRP labeling, washing, substrate loading and oil sealing for every digital sensor patch. Component 2 is then scanned on a fluorescent microscope for digital molecular counting at each array on the chip. Representative digital counting images at different concentrations are shown on the right. Unlabeled scale bar is 100 μm.

FIG. 3 (A) Calibration curves of the 3-plex DigiTACK. The limit of detection (LOD) value is calculated using the level of 3 standard deviations of blank signal plus the blank signal. The LODs of MCP1, IL6 and KC are calculated to be 0.576, 0.101, and 0.206 pg/mL respectively. Extra plots are generated for the large-dynamic-range calibration curves of MCP1 and KC since extremely high levels of these two markers are observed in devices after prolonged stimulation. (>10 hours) The increased linear dynamic range is achieved by reducing the labeling reagent concentrations during the “Labeling and Washing” step in FIG. 2D. (B) Analyte utilization rate plotted against the concentration of the measured solution. The utilization rate is defined as the fraction of the number of molecules captured by the sensor region to the total number of molecules present in the original bulk sample solution. Single molecule counting sensors utilize a minuscule number of analytes from the bulk solution, which is crucial to probing the microenvironment at high sensitivity. (C) Excellent measurement correlation (R2=0.9778) between the gold standard ELISA and the 3-plex DigiTACK measurements using the same cell secreted samples. (D)(i) Finite element analysis shows non-uniform concentration distribution across different regions in the bottom abluminal compartment after 2-hr of secretion and lateral diffusion. (ii) Finite element analysis demonstrates that an oscillating laminar flow homogenizes the abluminal compartment fluid. The concentration distribution becomes fairly uniform after just 4 oscillation cycles. (E) Concentration standard deviation of the 3 cut planes (shown in inset) versus the mixing duration. The standard deviation decreases with the mixing operation, quantitatively showing the effectiveness of the operation.

FIG. 4 (A) Phase contrast images of cultured or stimulated primary mouse brain microvascular endothelial cells (mBMEC) membrane tissues (i) Phase contrast image of basal mBMEC membrane tissue before the assay and (ii) after the assay. (iii) Phase contrast image of mBMEC membrane tissues before 500 ng/mL LPS stimulation and the assay and (iv) after 500 ng/mL LPS stimulation and the assay. No significant morphological alteration of mBMEC membrane tissues was observed after LPS stimulation and the DigiTACK assay. (B) Confocal microscopy images of mBMEC membrane tissue after the assay. Good quality cellular immunohistochemistry imaging and characterization can be performed after the Component 1 debonding and resealing processes. The green, red, and blue channels correspond to Claudin-5, ZO-1 and DAPI respectively. We observed slight defragmented staining of Claudin-5 and ZO-1 in LPS-treated groups compared with the control group after 10 hours. The dotted boxes mark the position of the zoomed-in images of a single channel or with overlayed channels.

FIG. 5 (A-F) Longitudinal 3-plex cytokine secretion monitoring of 8 arrayed devices. Each connected line represents the time variation of the targeted cytokine concentration in either the luminal or abluminal compartment of the same Component 1 throughout the assay period. 4 LPS-stimulated devices and 4 basal devices were monitored for up to 10 hours post stimulation/culture. Abluminal concentrations were measured with the integrated DigiTACK. Luminal concentrations were measured from supernatants collected at each timepoint during the assay then loaded and measured on the DigiTACK luminal measurement device. P-values between the two conditions were calculated at each timepoint for all 3 markers and we were able to capture significant differences between the LPS stimulated group and basal group even at the 2-hour time point for luminal MCP1, luminal KC and abluminal KC. (p-values=0.002, 0.04 and 0.02 respectively).

FIG. 6. Beadless digital counting sensor chip (Component 2) preparation procedure (I) Lithographic patterning photoresist on top of glass wafer. (II) Deep glass reactive ion etching of microwells. (III) Chemical vapor deposition of APTES into microwells. (IV) Stripping photoresist with acetone and apply hydrophobic coating to top surfaces. (V) Microfluidic patterning/conjugating of capture antibodies into microwells. (VI) Exfoliation of nonspecifically bound antibodies with low residue leaving tape. (VII) Finished hydrophilic-in-hydrophobic, capture-antibody-functioned microwell arrays for digital counting assays. Fluorescent top view of antibodies-functioned microwell arrays. Capture antibodies (rat-sourced) were labeled with Alexa fluor 488 anti-rat-IgG antibodies showing capture antibodies are specifically localized with high density within the microwells.

FIG. 7. Photo images of an exemplary DigiTACK platform. (A) 2×4 arrayed DigiTACK Component 1 strip. The 8 acrylic membrane housing blocks are adhered to a single PDMS thin film via PSA. The PDMS thin film has 8 arrayed through-cut channels at designated locations. (B) Etched DigiTACK Component 2 arrays with photoresist on top (FIG. 6 (II)). The photoresist acts as a mask to prevent APTES functionalization, further preventing unwanted antibody conjugation outside of the microwells. APTES and antibody conjugation outside of the microwells would render the surface hydrophilic, causing failure in the downstream oil sealing and compartmentation step in the digital assay. (C) Luminal sample measurement device for measuring multiple luminal compartment supernatants either collected from different devices or at different time points at high throughput. The device has a large area size with arrayed structural units. The device has the same bottom part as DigiTACK Component 2 with arrayed digital immunosensor patches. The device's top part has a two-dimensional array of soft-lithography-fabricated PDMS channels with sealed tops and inlets. All luminal supernatant samples are pipetted to the channels of the device via the inlets and measured at once.

FIG. 8. (A) Observed non-linearity of the KC calibration curve when the standards are prepared in the endothelial cell culture medium (ECM) purchased from Cell Biologics. The non-linearity phenomenon persists even after increasing the labeling concentration and incubation time, indicating that it is not caused by an insufficient incubation or labeling during the assay process. (B) Observed culture media-dependent signal non-linearity in KC calibration curves. When the standards are spiked in high glucose DMEM, excellent linearity is observed as opposed to ECM. Therefore, we switched the cell culture and stimulation media to DMEM after the cells grew and formed tight junctions in Component 1 of the DigiTACK platform.

FIG. 9. Experimental verification of the oscillation mixing efficiency. (A) Validation through food dye visualization. (i) Photo image of Component 1 of the DigiTACK platform. A food dye was introduced to the bottom channel from one port to create a non-homogeneous dye distribution. (ii) Photo image of the device after 5 oscillatory mixing cycles. The device was connected to a peristaltic pump, and 5 cycles of oscillatory flow mixing were applied. The flow rate was set at 1 μL/s, and the volume displacement of the oscillation was 5 μL per quarter cycle. (B) Instrument setup photo image of mixing 4 devices using a peristaltic pump. The programmable peristaltic pump was controlled through serial port communication with a laptop, and a MATLAB script programmed and ran the mixing cycles. (C) Calibration curve of a fluorometer for quantification of FITC concentrations. Excellent linearity was achieved. (D) Experimental result validating high mixing efficiency. No cells were cultured on top of the NPN membrane in this experiment. 1 mg/mL of FITC was added to the luminal compartment of 4 devices, and the abluminal channel was filled with PBS. FITC molecules were allowed to diffuse vertically through the nanoporous membrane and then diffuse laterally in the abluminal compartment for 40 minutes. 2 μL of supernatant was then extracted from abluminal compartment before and after oscillation mixing. Afterwards, the whole abluminal compartment volume was extracted. The plot shows the FITC concentrations of the extracted fluids. With the non-uniform distribution caused by restricted lateral diffusion in the abluminal compartment, the non-mixed samples extracted straight from the ports did not allow us to capture the concentration representing the abluminal compartment fluid. The non-mixed sample measurement yielded a CV as large as 49.9%. After mixing, the supernatant sample measurement resulted in a very small CV (1.8%) and obtained analyte concentration values very close to those from the full sample extraction measurement. This result indicates that sufficient fluid mixing was important even for small molecules like FITC with a larger diffusion coefficient.

FIG. 10. Cross-reactivity/specificity test of the 3-plex digital assay. DMEM samples were either spiked in with all 3 markers, spiked in with one marker, or not spiked in with any marker (control). The marker concentration of Ing/mL was used to assess the false positive and background. No significant cross-reactivity was observed. The y-axis is split into two sections for better visualization of the background.

### DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### DETAILED DESCRIPTION OF THE INVENTION

The present disclosure relates to systems, devices, and methods for using a first component (e.g., having a top reservoir and bottom reservoir that are fluidically connected through, and only through, a nanoporous membrane) that is able to be mated with a second component that comprises a plurality of microwells. Stimulating agents and/or candidate therapeutics may be added to the top reservoir such that cells (e.g., endothelial cells) in the top reservoir release one or more analytes that pass through the nanoporous membrane into the bottom reservoir (which may contain cells such as neurons) and into the microwells where they are captured and then detected.

Organ-on-a-chip platforms have the potential to offer more cost-effective, ethical, and human-resembling models than animal models for disease study and drug discovery. Particularly, the Blood-Brain-Barrier-on-a-chip (BBB-oC) has emerged as a promising tool to investigate several neurological disorders since it could provide a model of the multifunctional tissue working as an important node to control pathogen entry, drug delivery and neuroinflammation. A comprehensive understanding of the multiple physiological functions of the tissue model requires biosensors detecting several tissue-secreted substances in a BBB-oC system. However, current sensor-integrated BBB-oC platforms are only available for membrane integrity characterization based on permeability measurement. Protein secretory pathways are closely associated with the tissue's diseased conditions. At present, prior to the present disclosure, it is believed no biosensor-integrated BBB-oC platform permitting in situ tissue protein secretion analysis over time existed, which prohibits us from fully understanding the time-evolving pathology of a tissue barrier. Disclosed herein is a BBB-oC platform type named the “Digital Tissue-BArrier-CytoKine-counting-on-a-chip (DigiTACK chip),” which integrates digital immunosensors into BBB-oC and demonstrates multiplexed, ultrasensitive, longitudinal cytokine secretion profiling of individual cell cultures. In some embodiments, the integrated digital sensors utilize a beadless microwell format to perform an ultrafast “digital fingerprinting” of the analytes while achieving a low limit of detection (LOD) around 100-500 fg/mL for cytokines (e.g., mouse MCP1, IL6 and KC). The DigiTACK platform is extensively applicable to profile temporal cytokine secretion of other barrier-related organ-on-a-chip systems and can provide helpful insights in cytokine level fluctuations in sequentially controlled experiments.

The systems, devices, and methods find use with a wide variety of cell types. While particularly useful for analysis of cell types that provide biological barrier functions, the invention can be used with any cell types. The examples provided herein highlight the beneficial use to study and analyze BBB. The systems, devices, and methods also find use, for example, to analyze cells of associated with the intestinal epithelial barrier.

The present disclosure is not limited to particular biological molecules for inclusion in the microwells as analyte binding agent and is not limited by the labeling/detection agents employed. Examples include but are not limited to, proteins (e.g., antibodies), nucleic acid, lipids, or carbohydrates. In some embodiments, each of the microwells or arrays of microwells comprises one or more distinct analyte binding agent.

In some embodiments, the analyte binding agent comprises a detection moiety which can be directly detected. In some embodiments, the method may further comprise adding a labeling agent to the analyte binding agent, such that the labeling agent reacts with the detection moiety to produce a reaction product. Thus, in some embodiments, determining the presence or absence of the analyte detection agent comprises measurement of the reaction product. The labeling agent may be added before or after isolation of the analyte binding agent.

In some embodiments, the labeling agent is a substrate for an enzyme included in the analyte binding agent such that upon contact with the enzyme converts the labeling agent into a chromogenic, fluorogenic, or chemiluminescent reaction product, which is detectable. In some embodiments, the labeling agent is an enzyme and the substrate is included in the analyte binding agent. Any known chromogenic, fluorogenic, or chemiluminescent labeling agents may be selected for conversion by many different enzymes. In exemplary embodiments, the enzyme may be beta-galactosidase, horseradish peroxidase, or alkaline phosphatase. The substrate can respectively be an eta-galactosidase, horseradish peroxidase, or alkaline phosphatase well known in the art that are labeled or create a measurable signal upon enzymatic reaction, including, but not limited to: 3,3′,5,5′-tetramethylbenzidine, 3,3′-diaminobenzidine, 2,2′-azino-bis(3-ethylbenzothiazoline-6-sulphonic acid), p-nitrophenyl phosphate, 2,2′-azinobis [3-ethylbenzothiazoline-6-sulfonic acid]-diammonium salt, o-phenylenediamine dihydrochloride, or other enhanced fluorescent or chemiluminescent derivatives thereof.

The detection methods and type of detector employed depend on the nature of the analyte binding agent, detection agent, or labeling agent reaction products. Non-limiting examples of detection methods include optical imaging (fluorescence and visible), Raman scattering, spectroscopy (e.g., infrared, atomic, fluorescence or visible spectroscopies), absorbance, circular dichroism, electron microscopies (e.g., scanning electron microscopy (SEM), x-ray photoelectron microscopy (XPS)), light scattering, optical interferometry and other methods known in the art based on measuring changes in refractive index, diffraction, absorption, and fluorescence technologies.

In some embodiments, the detector may comprise more than one light source and/or a plurality of filters to adjust the wavelength and/or intensity of the light source. In some embodiments, the detector may also include a microscope (light or fluorescent) and/or a camera to capture the detection of the optical output of the detection method. The camera maybe a CCD (charge-coupled device) or CMOS (complementary metal-oxide-semiconductor) camera or similar camera known in the art. By using a camera with an electrical image converter, such as a CCD or CMOS chip, high local resolution can be achieved. The detector may also include a computer or controller used to control the light source, the filters, and/or execute any imaging processing software. The analyte binding agent may include different labels or detection chemistries, including for example, fluorescent, chemiluminescent, bioluminescent, or isotopic labels.

### EXAMPLES

### Example 1

### A Tissue-Barrier-on-a-Chip with Integrated Digital Immunosensors: In Situ Blood-Brain-Barrier Cytokine Secretion Monitoring

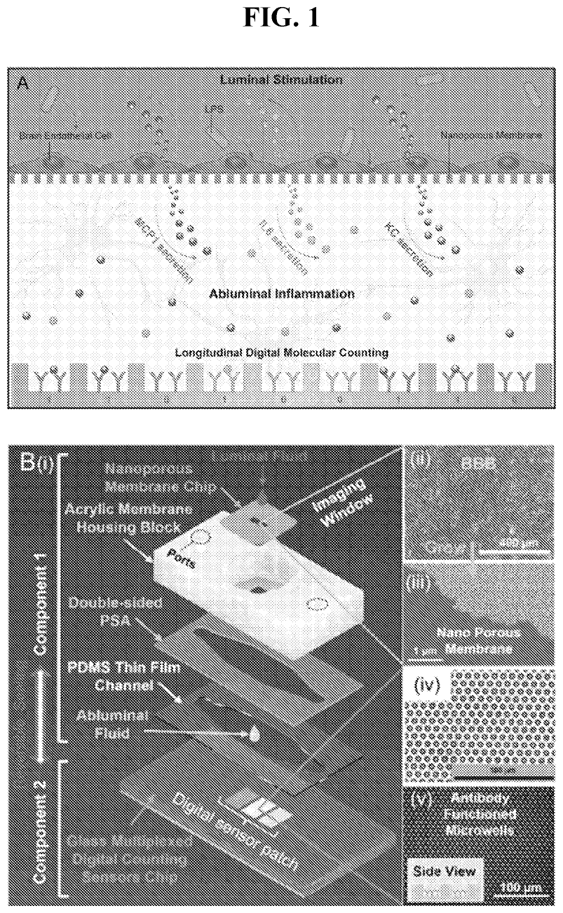

This example describes a new class of BBB-oC platform named “Digital Tissue-BArrier-CytoKine-counting-on-a-chip (DigiTACK)” to fill the technology gap described in the background. In this example, DigiTACK models a brain endothelial cell layer representing the BBB between the luminal (blood) and abluminal (brain) sides of the brain microvasculature that is subjected to endotoxin (LPS) exposure. The device detects a panel of three cytokine species relevant to neuroinflammation (FIG. 1A). In general, the DigiTACK device combines the μSIM platform (Castro Dias et al. 2021; Hudecz et al. 2020; Mansouri et al. 2022; McCloskey et al. 2022; Mossu et al. 2019; Salminen et al. 2020; all of which are herein incorporated by reference in their entireties, particularly for the uSIM platform) with an improved version of a previously reported single molecule-counting digital immunosensors (Song et al. 2020; Song et al. 2021a; Song et al. 2021b; Su et al. 2021; all of which are herein incorporated by reference in their entireties, particularly for single molecule counting digital immunosensors) (FIG. 1B).

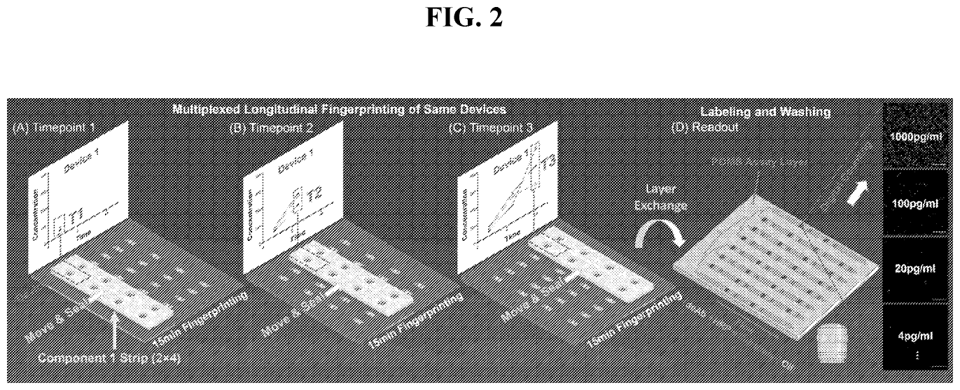

The uSIM platform features a highly permeable, bio-resembling ultrathin nanoporous silicon-nitride membrane with transparency for optical imaging. Its stacked Transwell™-like configuration (Mir et al. 2022, herein incorporated by references in its entirety, particularly for the bio chips disclosed therein) offers modularity and scalability. The integrated digital immunosensors have a multiplexed cytokine detection capability with a short incubation/detection time (<15 min) and a very low limit of detection (LOD) (~100-500 fg/mL). Each immunosensor only utilizes <0.1% of molecules in bulk solution to generate digital sensor signals, minimally affecting the analyte concentration in the original media during in situ measurements. All together, these immunosensor features enable one to perform in situ longitudinal cytokine secretion profiling for MCP1, IL6 and KC on both luminal and abluminal sides across a mouse brain microvascular endothelial cells (mBMEC) barrier formed on the DigiTACK chip. (FIG. 2). Stimulating the mBMEC barrier with endotoxin, we observed significant asymmetry in the cytokine secretion behavior of the layer between the luminal and abluminal sides and distinct, characteristic patterns with the temporal profiles across the three cytokines. These interesting observations highlight that the DigiTACK platform finds use for drug screening, disease mechanism exploration, and pharmacological therapy methods.

### Material and Methods

### DigiTACK Component 1 and Component 1 Strip Preparation

Component 1 is a modified version of the modular μSIM (McCloskey et al. 2022) which is composed of a nanoporous silicon nitride (NPN) membrane chip (SiMPore Inc., USA) (FIG. 1B(iii)), an acrylic membrane housing block (ALine Inc., USA), a through cut double-sided silicon-based pressure sensitive adhesive (PSA, ALine Inc., USA) and a through-cut Polydimethylsiloxane (PDMS) (DOW SYLGARD™ 184, Dow chemical, USA) thin film channel (FIG. 1B(i)). Details of the fabrication of the NPN membrane and acrylic membrane housing block as well as the assembly of these two parts can be found in a previously reported work (McCloskey et al. 2022, herein incorporated by reference for these components).

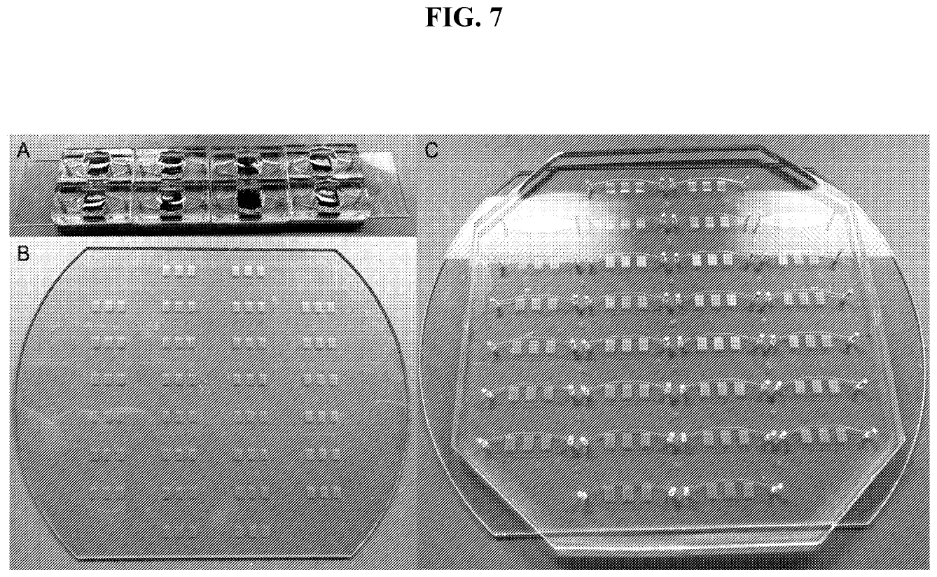

The PDMS thin-film channel layer was first prepared by spin coating uncured PDMS (curing agent to base ratio 1:10) on an acrylic substrate, curing it at 60° C., laser cutting both materials, then releasing the PDMS thin film channel from the acrylic substrate. Double-sided PSA layers with the same geometry as the PDMS thin film channels were also mass produced by laser cutting. We later irreversibly bonded the PDMS thin-film channel layer to the bottom of each acrylic housing block via the double-sided PSA layer. Multiple Component 1 units were assembled into a 2×4 arrayed strip-like format named “Component 1 strip” (FIG. 2A and FIG. 7A) for high-throughput experiments. The open-ended reservoir in the membrane housing block will later be referred to as the “luminal compartment”, and the bottom chamber enclosed by the PDMS thin film channel as the “abluminal compartment”.

### DigiTACK Component 2 (Beadless Digital Counting Chip) Preparation

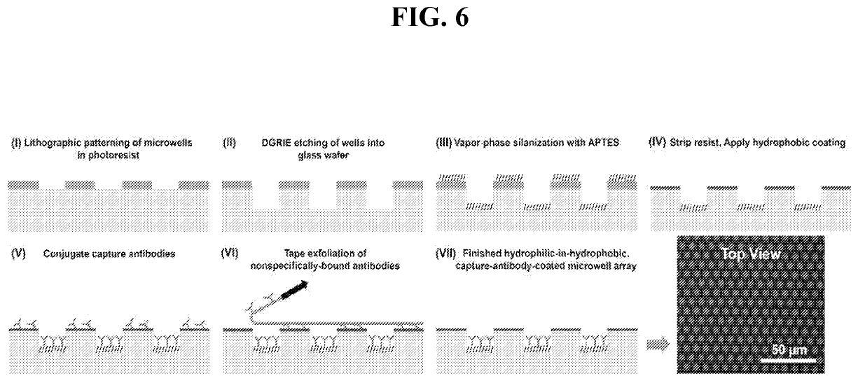

First, photoresist (MEGAPOSIT™ SPR™220, Dow chemical, USA) was patterned on a fused silica glass wafer using a conventional photolithography technique (FIG. 6(I)). Here, the patterned resist layer later serves as a mask for deep glass reactive ion etching (DGRIE) to etch microwells into the wafer (FIG. 6(II)). The wafer was then plasma activated and placed in a chemical vapor deposition (CVD) chamber for vapor phase APTES conjugations (FIG. 6(III)). The wafer was then sonicated in acetone for 5 minutes to remove photoresist and rinsed subsequently with isopropyl alcohol (IPA) and deionized water (DI) to remove acetone (FIG. 6(IV)). We then coated the glass wafer with PDMS to create a hydrophobic surface for oil sealing in the downstream digital assay. A PDMS layer with microfluidic channels (PDMS microfluidic channel layer) was then attached to the wafer to perform micropatterning and conjugation of capture antibodies. Then, a microfluidic spatial encoding method was applied to perform multiplexed digital assays in which a specific location was registered for the signal of a specific marker as used in previous studies (Song et al. 2021a; Song et al. 2021b; Su et al. 2021, all of which are herein incorporated by reference, particularly for multiplexed digital assays). MCP1 (#505901, BioLegend, USA), IL6 (#504501 BioLegend, USA), KC (#DY453-05, Bio-Techne, USA) capture antibodies were diluted in Phosphate-buffered saline (PBS) and incubated in designated microfluidic channels overnight. After antibody conjugation to the APTES (FIG. 6(V)), PBS-T (PBS+0.05% Tween 20) was flowed into the microfluidic channels to remove excess antibodies, and the PDMS microfluidic channel layer was removed. A low residue tape was then pressed on top of the microwells to exfoliate the nonspecifically bound antibodies that could not be removed through liquid phase washing (FIG. 6(VI)). These processes yielded a hydrophilic-in-hydrophobic capture-antibody-coated microwell array for digital counting assays as shown in FIG. 6(VII). For quality control, we further labelled the capture antibodies (rat-sourced) with Alexa fluor 488 anti-rat-IgG antibodies (#405418, BioLegend, USA) to verify that high-density conjugation of capture antibodies was successfully achieved within the microwells while leaving no antibodies on the microwells' top surfaces. The finished wafer is later referred to as Component 2 (FIG. 7B).

### Primary Mouse Brain Endothelial Cell Culture and Stimulation in DigiTACK

Primary mouse brain microvascular endothelial cells (mBMEC) (C57BL/6 mouse primary brain microvascular endothelial cells, #C57-6023, Cell Biologics, USA) were cultured in endothelial cell medium (ECM) (#M1168 Complete Mouse Endothelial Cell Medium, Cell Biologics, USA). mBMEC were seeded onto 0.5% gelatin-coated flasks and cultured in endothelial cell medium (ECM) supplemented with 20% Fetal bovine serum (FBS), 1% antibiotic-antimycotic solution, growth factors cocktails (VEGF, ECGF and EGF), hydrocortisone and heparin (#M1168 Complete Mouse Endothelial Cell Medium, Cell Biologics, USA) at 37° C. and 5% CO₂ in humid atmosphere.

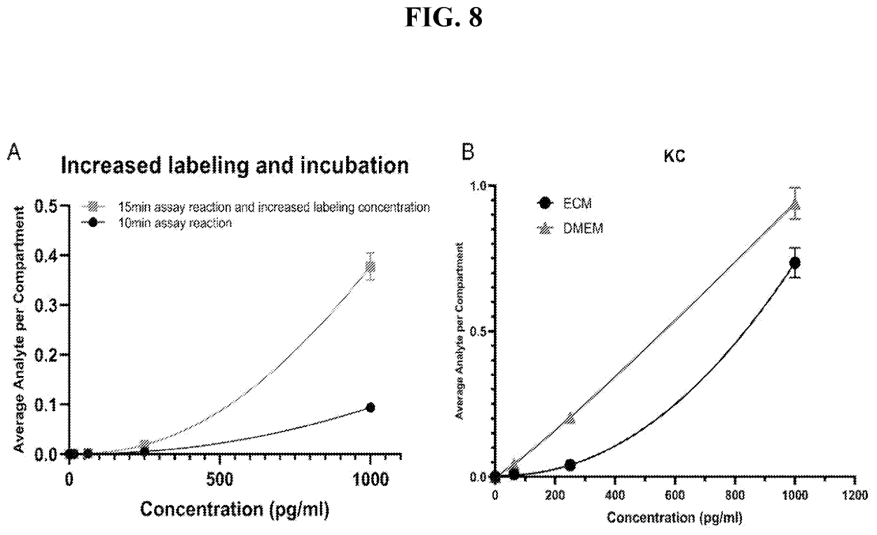

In the cell preparation phase of the experiment, a Component 1 strip was first attached and sealed to a glass surface without digital sensor patches. The NPN membranes in the Component 1 strip were first coated with a gelatin-based coating buffer (#6950 Gelatin-Based Coating Solution, Cell Biologics, USA) for >30 minutes at 37° C. Then, we plate 100 μL of cell suspension at concentrations 1.4×10⁵ cells/mL onto the NPN membrane in the luminal compartment of each device (~480 cells/mm²) and fill the abluminal compartment with just the ECM. The devices are then incubated in a moisture controlled, 37° C., 5% CO₂ incubator. We replaced culture medium every other day with preheated ECM. The cultured mBMEC usually formed tight junctions within 3 to 4 days post-plating and became ready for endotoxin stimulation in the DigiTACK assay. For the stimulated groups, we dispensed freshly prepared and prewarmed LPS (500 ng/mL) in high glucose Dulbecco's Modified Eagle Medium (DMEM) into the luminal compartment at the beginning of the assay while filling abluminal compartment with pre-warmed high glucose DMEM. For the basal/control groups, cells were exposed to prewarmed high glucose DMEM in both the luminal and abluminal compartments. We chose high glucose DMEM as the diluent in the stimulant because we observed a non-linear digital sensor signal response in the spike-in KC calibration curves when using ECM (FIG. 8). The non-linearity is possibly due to the matrix effect and the interference of multiple growth factors that are added in the ECM.

### DigiTACK Longitudinal Assay and Readout

At the first time point of interest after LPS stimulation, the DigiTACK longitudinal assay started with moving the Component 1 strip to a location on Component 2 where each abluminal compartment fluid was paired with and exposed to a set of digital sensor patches (FIG. 2A). Here, pressure was applied to ensure the sealing between the PDMS layer of the Component 1 strip and the glass substrate of Component 2. Recording the cytokine concentrations in the abluminal compartment fluid requires the fluid to be in contact with the digital sensor patches only for 15 minutes, which we refer to as “pre-equilibrium digital fingerprinting.” At the end of the pre-equilibrium digital fingerprinting process, the Component 1 strip was moved to another sensor-free location on Component 2. At the subsequent time points, we repeated the assay procedure, which involved moving the strip to a location with a new set of digital sensor patches and pre-equilibrium digital fingerprinting (FIG. 2A, 2B, 2C). Component 2 currently incorporates 28 sets of digital sensor patches. Employing multiple Component 2 chips can further expand the assay's capacity to accommodate a larger number of digital fingerprinting time points or conditions than tested in this study. At the end of the longitudinal assay, the Component 1 strip was removed from Component 2 and attached to a thin microscope coverslip for downstream tissue fixation and immunofluorescent staining/imaging. Component 2 was subsequently attached to a PDMS assay layer (FIG. 2D) for downstream high-throughput digital immunosensor signal readout. The signal readout process involves detection-antibody labelling (MCP1 (#506002, BioLegend, USA), IL6 (#504601 BioLegend, USA), KC (#DY453-05, Bio-Techne, USA)), horseradish peroxidase (HRP) labeling, washing (5 minutes after detection antibody and HRP labeling in PBS+0.05% Tween 20), substrate loading (QuantaRed™ Enhanced Chemifluorescent HRP Substrate, Thermo Fisher, USA), and sealing with fluorocarbon oil (Novec™ 7500, 3M, USA) to physically isolate and compartmentalize each microwell from others. Component 2 attached to the PDMS assay layer was then placed on the motorized stage of a fluorescent microscope. We pre-programmed the motorized stage such that it scanned every digital sensor patch on Component 2.

During the digital immunosensor signal readout process, a QuantaRed fluorescent channel (545 nm/605 nm, excitation/emission) was used to count the “on” microwell spots at each sensor patch. The number of the “on” spots directly correlates to the target analyte's concentration (FIG. 2D). Scanning the entire images of the digital immunosensor microwells on Component 2 took less than 10 minutes. Standards for mouse MCP1, IL6 and KC to generate DigiTACK calibration curves were all obtained from R&D system's DuoSct ELISA kits (MCP1: #DY479, IL6: #DY40605, KC: #DY45305). We used ELISA to validate the DigiTACK measurements of MCP1, IL6 and KC. The MCP1 and IL6 ELISA measurements were performed by the standard plate coating and assay protocol using antibody pairs from BioLegend mentioned above. KC ELISA measurements were done with R&D systems Mouse KC DuoSet ELISA kits (#DY453-05, Bio-Techne, USA).

### Data Analysis by a Convolutional Neural Network

We process all scanned digital immunosensor images using a previously reported convolution neural network (CNN) algorithm. The algorithm runs two signal recognition pathways in parallel. One CNN recognizes and counts the enzyme active “On” microwells and the other recognizes defects and contaminations. The algorithm starts from a pre-processing process, including image cropping, contrast enhancement, noise filtering, light source uniformity and background correction. The CNN then classified each image pixel into two categories: (1) image defects and (2) background. The “On” microwells counted by ImageJ were segmented out as the output mask with defects removed. More details in the machine learning algorithms can be found in our previously reported paper (Song et al. 2021b, herein incorporated by reference, particularly for the machine learning algorithms). Lastly, the fraction of the “On” microwells with respect to the total microwells (Pon) was calculated, and the Poisson's distribution equation was used to calculate the mean expectation value: λ=−ln (1−Pon), which represented the average number of analyte per compartment (AAC) and is proportional to the concentration of the analyte (Zhang and Noji 2017).

### Immunofluorescent Staining

At the end of the DigiTACK longitudinal assay, the Component 1 strip was detached from Component 2, and it was resealed on top of a microscope slide cover slip (Fisherbrand 24×60-1, Thermo Fisher, USA). The mBMECs cultured in the Component 1 strip were then fixed with 4% paraformaldehyde for 15 minutes at room temperature (RT), followed by three 5-minute washes with phosphate-buffered saline (PBS). The luminal compartments were later incubated in blocking solution (0.05% TritonX-100, 5% normal goat serum, 1×PBS) for one hour at RT. Then, we added fluorescently labelled primary antibodies targeting ZO-1 and Claudin-5 (#352588, #339194, Thermo Fisher, USA)(dilution of 1:100 in blocking solution) to the sample and incubated at 4° C. overnight. The devices were washed with PBS and incubated with DAPI (1:500 dilution in PBS) for 2 hours at RT. After the PBS washing process, the mBMEC tissues in the Component 1 strip became ready for fluorescent or confocal microscopy.

### COMSOL Finite Element Analysis

We assumed a constant cytokine flux into the bottom abluminal channel fluid across the membrane during the secretion period. The flux value was back calculated from the endpoint measurements of the analytes. Using the flux value, the KC concentration distribution in the bottom abluminal channel was simulated after 2 hours of secretion, where the simulation accounted for the lateral diffusion of the analyte molecules entering the channel through the membrane pores. The simulation solved the convection-diffusion equation (Eq. 1).

$$\partial c / \partial t = ∇ \cdot ( D ∇ c ) - ∇ \cdot ( uc ) + R \qquad ( Eq . 1 )$$

where c is the analyte concentration, t is time, D is the analyte diffusion coefficient, u is the velocity of the abluminal channel fluid in convection, and R is the analyte generation (annihilation) rate of the source (sink) in the system. The value of D was obtained from the Stokes-Einstein equation (Eq. 2), and the analyte's Stokes radius R was estimated by Eq. 3.

$$D = kT / 6 πη R \qquad ( Eq . 2 )$$

$$R = \sqrt[3]{\frac{3 M W}{4 π {N}_{A}} υ} \qquad ( Eq . 3 )$$

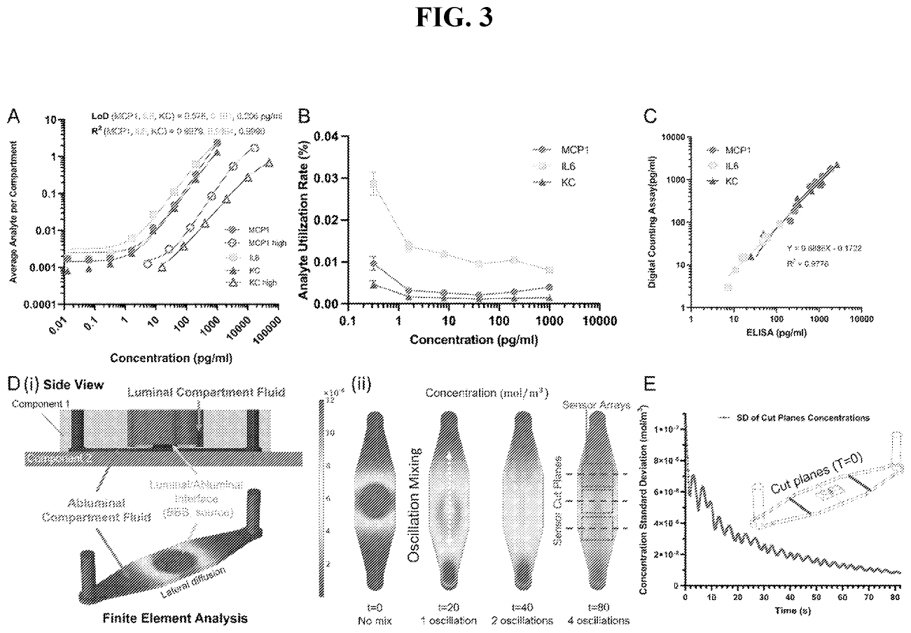

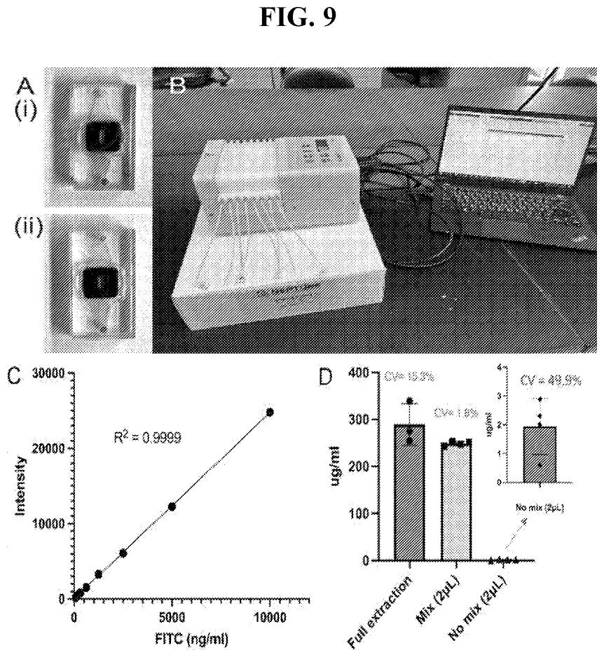

Here, the k is the Boltzmann constant, T is the temperature, n is the viscosity of the abluminal channel fluid, and R is the Stokes radius of the analyte molecule. To use Eq. 3, we assumed a spherical structure with closely packed atoms for the KC molecule. Here, MW is the molecular weight of KC, NA is the Avogadro's number, and v is the partial specific volume of KC, which was estimated to be the typical value for proteins, 0.73 g/cm3 (Erickson 2009). These estimated values were used in our finite element analysis by COMSOL to predict the post-secretion KC concentration distribution (FIG. 3D(i), Video 6). Subsequently, we simulated the transient KC distribution under convective mixing of the abluminal channel fluid. The convective mixing was generated by oscillating the directions of a laminar inflow through the abluminal channel inlet/outlet ports under no-slip boundary conditions. High mixing efficiency of the simulated process was experimentally validated as shown in FIG. 9.

### Statistics

All error bars in the graphs shown represent a standard deviation unless otherwise specified. Group analysis was all performed using an unpaired Welch's t test, and a p-value of <0.05 was considered as statistically significant. The digital immunosensor assay standard curves were fitted with a 4-parameter logistic regression by GraphPad Prism 9 (GraphPad Software, USA). The LOD of every marker was determined by the signal corresponding to three times the standard deviation above the average blank signal.

### Results

DigiTACK Assay with Multiplexed Beadless Digital Counting Sensors

DigiTACK, in certain embodiments, is a reversibly sealable BBB-oC microfluidic device that includes two components. The top component (Component 1) composed of two fluidic compartments (luminal and abluminal) separated by a brain endothelial cell (mBMEC) barrier grown on top of a NPN membrane (FIG. 1B(i)). The bottom component (Component 2) is, in certain embodiments, a beadless digital counting glass chip fabricated by conventional lithography patterning, etching, and novel exfoliation-based antibody patterning to achieve high-density antibody immobilization within microwells (FIG. 6). An array of three rectangular patterns (89,760 microwells) forms one of the digital sensor patches for profiling one abluminal compartment in Component 1 (FIG. 1B(i), 1B(iv), 1B(v)). We pair each digital sensor patch patterned on a location of the Component 2 glass chip with one of the abluminal compartments in the Component 1 strip. Each digital sensor patch location is specifically assigned to detect the concentration profiles of three different protein analytes in the abluminal compartment fluid at a different assay time point or under different assay conditions (FIG. 2). The arrayed DigiTACK configuration enables high-throughput profiling of mBMEC tissue-secreted cytokines (or human tissue-secreted cytokines if human cells are employed). The DigiTACK longitudinal measurement in this study involves moving the Component 1 strip from one location to another on Component 2 (FIG. 2A, 2B, 2C). Upon the completion of the longitudinal measurement, the digital senor patches on Component 2 retain all the analyte concentration information obtained at different time points. This information is read out from the parallelly labelled digital sensors on Component 2 via single-molecule digital counting (FIG. 2D).

### Characterization of DigiTACK Performance

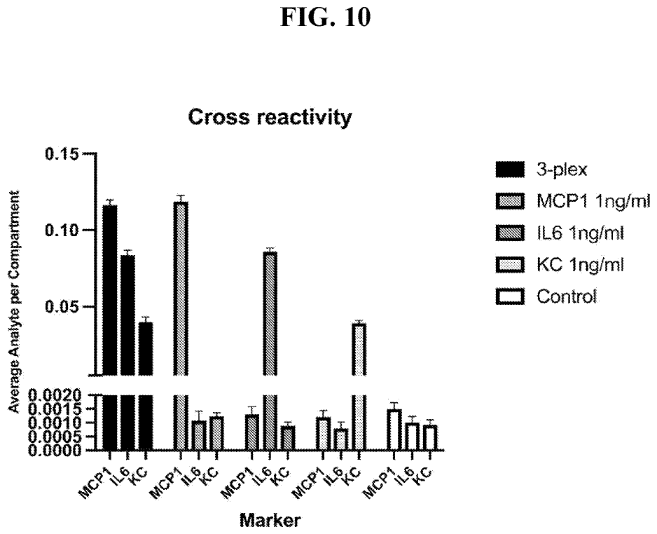

Using high glucose DMEM spiked by known concentrations of MCP1, IL6 and KC, we obtained standard curves for these three analytes and determined their LOD values of 0.576, 0.101, 0.206 pg/mL, respectively (FIG. 3A). The whole standard curve acquisition process for the three analytes required only 15 minutes. From preliminary experiments with LPS stimulated endothelial cells, we observed that after 10 hours of secretion, MCP1 and KC concentrations reached up to ~6000 and 20000 pg/mL levels, respectively. Therefore, we decreased the concentrations of the MCP1 and KC sensor's detection antibody and the HRP label to maintain signal linearity at high concentrations for prolonged stimulation experiments. Overall, we obtained ultrahigh sensitivity and a linear dynamic range of ~4 orders of magnitude (~0.1 pg/mL to ~1000 pg/mL) for the DigiTACK immunosensors, and we could extend the linear dynamic range by 1~2 orders of magnitude with the lowered labelling efficiency. Additionally, the beadles assay format used in the assay could further improve the sensor's linear dynamic range. Directly immobilizing capture antibodies within microwell compartments, this method eliminates the stochastic process of beads settling into the microwells by gravity, which is typically employed in the conventional bead-based digital immunoassay yielding a ~55% microwell filling rate (Song et al. 2021b). As a result, our method can achieve a 100% microwell utilization rate, suppressing sensor signal saturation and offering a larger digital linear dynamic range for a given sensor size. Notably, we did not observe any cross-reactivity between the three markers utilizing this beadless format (FIG. 10).

In the DigiTACK measurement, the digital immunosensors uptake only a miniscule portion of the analytes from the assayed solution. We first define the “analyte utilization rate” as the fraction of the number of the analyte molecules taken for the measurement to the total number of the molecules present in the original sample fluid. The Poisson statistics-derived average-analyte-per-compartment (2) times the total number of compartments in the sensors represents the total number of the molecules captured by a single digital sensor patch. We further divide the total captured molecules by the total number of molecules in the assayed sample volume, providing an average utilization rate (FIG. 3B). The utilization rate from 0.3 pg/mL to 1000 pg/mL are all well below 0.1% for all three markers. Thus, the sensors do not alter the analyte concentration of the bulk sample fluid even when operating at low concentration levels. The gradual increase of the utilization rate with the decreasing concentration is due to the sensor signals approaching the nonspecific binding of HRP background, not a greater proportion of analyte uptake (FIG. 3B). We measured the same cell secreted markers using both the gold standard ELISA and the 3-plex DigiTACK assay. We found excellent correlation between the two assay methods (R2=0.9778) (FIG. 3C). From the linear fitting formula, the slope is 0.8888, which indicates the high accuracy of the 3-plex DigiTACK assay compared to the gold standard single-plex ELISA.

The experiment is performed under the condition where the tissue-secreted analytes are measured within a microfluidic device experiencing little bulk fluid flow or mixing during the tissue culture experiment. We then examined how analyte exposure to the digital sensor patches would reflect the total analyte content in the abluminal compartment fluid under such a condition. Finite element analysis was performed on the abluminal compartment fluid as indicated in FIG. 3D (i). The result shows that the concentration is highly non uniform, and mixing is needed for accurate and reproducible multiplexed measurements by the spatially encoded digital sensors. We therefore further modelled the concentration profile after oscillatory mixing events using multiple laminal inflows through the ports (FIG. 3D (ii)). We observed that the geometry of the bottom abluminal channel can create sufficient homogenizing effects through the oscillating flows. With only 4 oscillation cycles, the concentrations on top of the sensor areas become nearly the same as each other. We plotted the standard deviation of the averaged surface concentration on three sensor cut planes (shown in the inset of FIG. 3E) throughout the mixing period and quantitatively characterized the mixing efficiency (FIG. 3E). The standard deviation decreases and approaches 0 after just a few cycles. For consistency, we applied 20 cycles of oscillation mixing to all devices at every measurement time point using a programmed high throughput 8-channel peristaltic pump (Experimental verification of mixing efficiency shown in FIG. 9).

Primary Mouse Brain Microvascular Endothelial Cell Barrier Imaging with DigiTACK

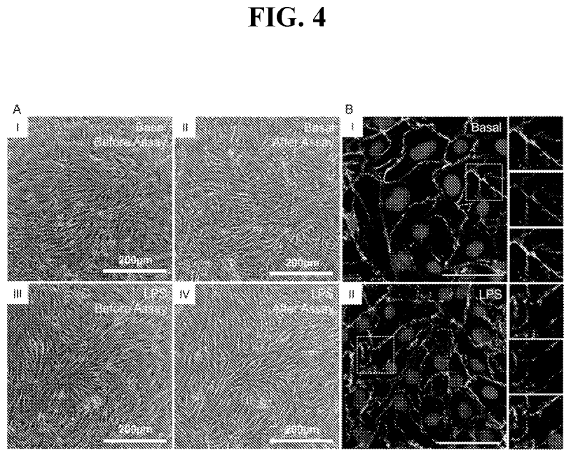

Primary mouse brain microvascular endothelial cells (mBMECs) were cultured to confluence and formed tight junctions on NPN membrane in the DigiTACK device (FIG. 4A (I), 4A(III)). We did not observe significant morphological differences before and after the DigiTACK longitudinal assay involving the repeated relocation and bonding/debonding of the Component 1 strip (FIG. 4A (II), 4A(IV)). This can be attributed to the ability of the NPN membrane to hydraulically decouple the top luminal and bottom abluminal compartments. Across the membrane, diffusion is seamless while incidental convection is stopped by the high flow resistance of the 60 nm pores. Therefore, cells on the membrane are protected despite being <100 nm away from the fluidic disturbances in the abluminal compartment (Chung et al. 2014). Furthermore, we performed immunofluorescent staining on tight junction proteins ZO-1 (Alexa Fluor 594) and Claudin-5 (Alexa Fluor 488). In all samples, continuous ZO-1 and Claudin 5 staining was detected at the borders of the mBMECs, indicating the presence of an intact and functional barrier. We observe slight morphological changes in junctional protein Claudin-5 and ZO-1, manifested as defragmented staining, in LPS-treated groups compared with the control group (FIG. 4B(I) vs. 4B(II)). The qualitative nature of the immunohistochemistry provides some level of understanding of the cultured tissue's functions. But it does not capture quantitative signature differences in the cell phenotype under these stimulation conditions, which again highlights the need of an integrated immunosensor in BBB-oC platforms.

### Longitudinal Multiplexed Protein Secretion Monitoring of Endothelial Barriers

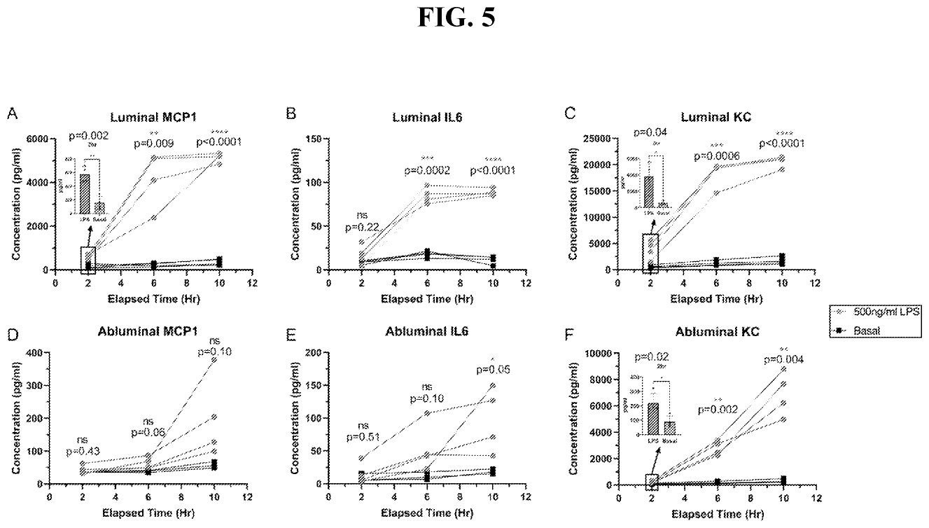

Using the DigiTACK platform, we measured temporal concentration changes of the 3 cytokine markers in a Component 1 strip at three time points within 10 hours of stimulation/culture (2-hour, 6-hour, and 10-hour) (FIG. 5). The cytokine markers in the abluminal compartment fluid were measured in situ using the integrated digital immunosensors as described in the Materials and Methods section. The large sample volume >100 μL held in the top luminal compartment in Component 1 allowed sequential collection of a 10 μL supernatant with a multichannel micropipette during the assay without altering the original luminal cytokine concentrations. Therefore, all luminal compartment fluid concentrations were measured on a separate DigiTACK luminal measurement device (FIG. 7C), which shares the same Component 2 with DigiTACK, allowing measurements of all coordinating luminal concentrations in one batch of experiment.

For the luminal-side data in FIGS. 5A, 5B and 5C, we observed a significant cytokine secretion response following the 500 ng/mL LPS stimulation. All three markers appeared to plateau at around the 10-hour time point with MCP1 and KC approaching ~6000 pg/mL and ~20000 pg/mL, respectively. The IL6 level stabilized at ~100 pg/mL for the stimulated groups. All basal devices showed low cytokine secretion throughout the longitudinal experiment. Interestingly, we were able to capture significant differences between the stimulated and basal groups even at 2 hours for MCP1 (p=0.002) and KC (p=0.04). Although IL6 also appeared to begin rising as soon as 2 hours after stimulation, the variability in secretion was much greater. After 2 hours, all 3 markers were significantly elevated by LPS stimulation.

For the abluminal-side data in FIGS. 5D, 5E and 5F, we found that cytokine secretion was lower and also more variable than in the luminal compartment. In basal conditions, abluminal cytokine secretion remained low during 10 hours of culture. MCP1 and IL6 concentrations were both above basal conditions 6 hours after stimulation, although high variability precluded statistically significant differences from controls (p=0.06 and 0.1 respectively). Abluminal KC, however, showed a linear trend throughout the 10-hour LPS stimulation period and even showed significant differences at the 2-hour time point (p=0.02, stimulated average 219.2 pg/mL vs. basal average 90.8 pg/mL). At the 10-hour time point, the stimulated KC concentration reached an average value of 6920.3 pg/mL and the trend showed no sign of saturation.

The data show that, within the same marker, there are significant concentration gradients between the luminal and abluminal side of the endothelial barrier. At 10 hours, we see gradients as large as ~5000 pg/mL between the barrier (~25-fold differences) for the MCP1, and ~13700 μg/mL for KC (~3-fold differences). The stimulation on one side of the barrier may have induced some level of polarization of the brain endothelial cells, resulting in more secretion events at the stimulated luminal side than the abluminal side.

Capturing cytokine secretion profiles in BBB-oC systems is critical in assessing CNS therapeutic side effects and understanding disease mechanisms relating to neuroinflammation caused by the infiltration of bloodborne cells or substrates (e.g., multiple sclerosis, encephalitis, strokes). The literature has reported the roles of cytokines in neurological disorders either as factors that induce leukocyte infiltration, or as factors that more directly cause neural tissue damage. However, in-depth understanding of these disease progressions remains unknown either due to the difficulty of separating variables in complex in vivo models or the current insufficient and granular data restricted by end-point external measurements from previous BBB-oC platforms.

To address the challenge above, we have developed a BBB-oC platform to feature integrated immunosensors named “DigiTACK.” Permitting a novel beadless microwell digital assay within the BBB-oC, DigiTACK offers ultrasensitive multiplexed abluminal cytokine concentration measurements with LODs of a few hundreds of fg/mL while minimizing the assay time and maintaining an excellent correlation with the gold standard ELISA measurements. Additionally, the DigiTACK assay simultaneously allows for real-time brightfield imaging and end-point immunohistochemistry characterization of the BBB. We designed the reversible “tacking and fingerprinting” strategy for the abluminal cytokine measurements to address several difficulties and requirements associated with immunosensing in the BBB-oC platform. First, the small sample volume (<20 μL) of the abluminal compartment on each tissue culturing device prohibits supernatant extraction from the sample. If the measurement was carried out ex situ, the assay would require diluting the supernatant and replenishing the culture medium, which result in sensitivity loss and BBB microenvironment alterations. In contrast, implementing the digital fingerprinting technique here allows for dilution-free low-concentration analyte detection without altering the abluminal microenvironment. Second, a previous study (Naegelen et al. 2015) indicates that analyte concentrations in a cell-containing environment can decrease during a longitudinal experiment due to the cellular intake and degradation of analytes. Resetting or renewing the sensors at each measurement time point is crucial to capture these concentration fluctuations. The reversible “tacking and fingerprinting” assay achieves this by moving and exposing an array of tissue culture devices to a new set of digital sensor patches at each time point.

In this example, we used a simplified LPS-stimulated mBMEC barrier model in the DigiTACK platform. Even the simple model allowed us to observe several interesting phenomena, such as (i) various concentration dynamic ranges and secretion rates for different cytokine markers, (ii) distinct cytokine responsiveness and homeostasis time points between the different sides of the barrier (i.e., secretion polarization), and (iii) marker-dependent gradient magnitudes across the mBMEC barrier. The secretion level was observed to approach steady state at about 6 hours on the luminal side. In contrast, the concentrations of all three markers continued to climb on the abluminal side. This suggests that the abluminal cytokine secretion may experience a relatively delayed effect while the luminal secretion manifests a fast and drastic response. The linearly increasing KC levels on the abluminal side may eventually approach the luminal compartment concentration. While not necessary to understand or practice the present invention, it is speculated that the KC concentration parity between the two sides may compel neutrophils to migrate across the endothelial barrier (Salminen et al. 2020). This could be tested by more experiments with even longer stimulation to see if the abluminal side eventually reaches steady state and determine at what concentration levels it occurs.

It is believed that this example is the first to provide sequential temporal in situ profiling of multiple cytokines/chemokines. It indicates that the DigiTACK may find wide use in recording abrupt cytokine secretion changes in response to a stimulus or cells introduced in sequentially controlled experiments. For example, for multiple sclerosis or stroke studies, DigiTACK could facilitate parallel monitoring of cytokine concentration fluctuations upon observing or introducing immune cell infiltrations (Lopes Pinheiro et al. 2016). For dynamic BBB and CNS studies, by sequentially altering luminal fluids and removing stimulants, one could use the DigiTACK to capture the timing when the abluminal side of the BBB returns to its homeostatic cytokine concentration (DiSabato et al. 2016; Gilroy and Lawrence 2008). The low molecular utilization rate and short pre-equilibrium profiling time of the integrated digital immunosensors proves the ultrahigh sensitivity and minimal invasiveness of the DigiTACK.

Using these remarkable device features, one could probe rapid non-classical cytokine secretory pathways that have been impossible to study with conventional methods. Such secretory pathways can be studied by measuring preformed cytokines' immediate release from granules (e.g., neutrophils) into the local microenvironment within minutes of receptor stimulation (Lacy and Stow 2011). In some embodiments, the DigiTACK device feature is further expanded with a flow module integrated in the top luminal reservoir for dynamic circulation and shear force modeling (e.g., Mansouri et al. 2022), human iPSC-derived pericytes, astrocytes and microglia co-cultured in the bottom abluminal channel for neuroinflammation analysis under a more real BBB microenvironment, and highly integrated miniaturized immunosensors offering increased multiplexity for high throughput proteomics studies.

In summary, the various embodiments of DigiTACK demonstrate utility in sequential multiplexed cytokine profiling in an organ-on-a-chip. While this example was specifically aimed at validating the DigiTACK's ability to characterize the secretory behavior of a mBMEC barrier for future BBB studies, the DigiTACK can be applied to any barrier-related organ-on-a-chip systems by switching the cultured barrier cell types. With further adoptions of human iPSCs into the platform, the DigiTACK platform can be a versatile platform to facilitate multiple human CNS disease model progression studies, and ultimately leading to OoC-guided, personalized precision medicine therapeutics.

### REFERENCES

Ahn, S. I., Sei, Y. J., Park, H. J., Kim, J., Ryu, Y., Choi, J. J., Sung, H. J., MacDonald, T. J., Levey, A. I., Kim, Y., 2020. Microengineered human blood-brain barrier platform for understanding nanoparticle transport mechanisms. Nat Commun 11 (1), 175.
Badiola-Mateos, M., Di Giuseppe, D., Paoli, R., Lopez-Martinez, M. J., Mencattini, A., Samitier, J., Martinelli, E., 2021. A novel multi-frequency trans-endothelial electrical resistance (MTEER) sensor array to monitor blood-brain barrier integrity. Sensors and Actuators B: Chemical 334.
Carbone, L., 2021. Estimating mouse and rat use in American laboratories by extrapolation from Animal Welfare Act-regulated species. Sci Rep 11 (1), 493.
Castro Dias, M., Odriozola Quesada, A., Soldati, S., Bosch, F., Gruber, I., Hildbrand, T., Sonmez, D., Khirc, T., Witz, G., McGrath, J. L., Piontek, J., Kondoh, M., Deutsch, U., Zuber, B., Engelhardt, B., 2021. Brain endothelial tricellular junctions as novel sites for T cell diapedesis across the blood-brain barrier. J Cell Sci 134 (8).
Chung, H. H., Chan, C. K., Khire, T. S., Marsh, G. A., Clark, A., Jr., Waugh, R. E., McGrath, J. L., 2014. Highly permeable silicon membranes for shear free chemotaxis and rapid cell labeling. Lab Chip 14 (14), 2456-2468.
DiSabato, D. J., Quan, N., Godbout, J. P., 2016. Neuroinflammation: the devil is in the details. J Neurochem 139 Suppl 2, 136-153.
Erickson, H. P., 2009. Size and shape of protein molecules at the nanometer level determined by sedimentation, gel filtration, and electron microscopy. Biol Proced Online 11, 32-51.
Gilroy, D., Lawrence, T., 2008. The resolution of acute inflammation: A ‘tipping point’ in the development of chronic inflammatory diseases. In: Rossi, A. G., Sawatzky, D. A. (Eds.), The Resolution of Inflammation, pp. 1-18. Birkhäuser Basel, Basel.
Hudecz, D., Khire, T., Chung, H. L., Adumeau, L., Glavin, D., Luke, E., Nielsen, M. S., Dawson, K. A., McGrath, J. L., Yan, Y., 2020. Ultrathin Silicon Membranes for in Situ Optical Analysis of Nanoparticle Translocation across a Human Blood-Brain Barrier Model. ACS Nano 14 (1), 1111-1122.
Lacy, P., Stow, J. L., 2011. Cytokine release from innate immune cells: association with diverse membrane trafficking pathways. Blood 118 (1), 9-18.
Larochelle, C., Alvarez, J. I., Prat, A., 2011. How do immune cells overcome the blood-brain barrier in multiple sclerosis? FEBS Lett 585 (23), 3770-3780.
Lecuyer, M. A., Kebir, H., Prat, A., 2016. Glial influences on BBB functions and molecular players in immune cell trafficking. Biochim Biophys Acta 1862 (3), 472-482. Liang, Y., Yoon, J.-Y., 2021. In situ sensors for blood-brain barrier (BBB) on a chip. Sensors and Actuators Reports 3.
Lombardi, A., Cantini, G., Mello, T., Francalanci, M., Gelmini, S., Cosmi, L., Santarlasci, V., Degl'Innocenti, S., Luciani, P., Deledda, C., Annunziato, F., Forti, G., Galli, A., Serio, M., Luconi, M., 2009. Molecular mechanisms underlying the pro-inflammatory synergistic effect of tumor necrosis factor alpha and interferon gamma in human microvascular endothelium. Eur J Cell Biol 88 (12), 731-742.
Lopes Pinheiro, M. A., Kooij, G., Mizee, M. R., Kamermans, A., Enzmann, G., Lyck, R., Schwaninger, M., Engelhardt, B., de Vries, H. E., 2016. Immune cell trafficking across the barriers of the central nervous system in multiple sclerosis and stroke. Biochim Biophys Acta 1862 (3), 461-471.
Low, L. A., Mummery, C., Berridge, B. R., Austin, C. P., Tagle, D. A., 2021. Organs-on-chips: into the next decade. Nat Rev Drug Discov 20 (5), 345-361.
Lv, S., Song, H. L., Zhou, Y., Li, L. X., Cui, W., Wang, W., Liu, P., 2010. Tumour necrosis factor-alpha affects blood-brain barrier permeability and tight junction-associated occludin in acute liver failure. Liver Int 30 (8), 1198-1210.
Mansouri, M., Ahmed, A., Ahmad, S. D., McCloskey, M. C., Joshi, I. M., Gaborski, T. R., Waugh, R. E., McGrath, J. L., Day, S. W., Abhyankar, V. V., 2022. The Modular microSIM Reconfigured: Integration of Microfluidic Capabilities to Study In Vitro Barrier Tissue Models under Flow. Adv Healthc Mater, e2200802.
McCloskey, M. C., Kasap, P., Ahmad, S. D., Su, S. H., Chen, K., Mansouri, M., Ramesh, N., Nishihara, H., Belyaev, Y., Abhyankar, V. V., Begolo, S., Singer, B. H., Webb, K. F., Kurabayashi, K., Flax, J., Waugh, R. E., Engelhardt, B., McGrath, J. L., 2022. The Modular microSiM: A Mass Produced, Rapidly Assembled, and Reconfigurable Platform for the Study of Barrier Tissue Models In Vitro. Adv Healthc Mater 11 (18), e2200804.
Mir, M., Palma-Florez, S., Lagunas, A., Lopez-Martinez, M. J., Samitier, J., 2022. Biosensors Integration in Blood-Brain Barrier-on-a-Chip: Emerging Platform for Monitoring Neurodegenerative Diseases. ACS Sens 7 (5), 1237-1247.
Mossu, A., Rosito, M., Khire, T., Li Chung, H., Nishihara, H., Gruber, I., Luke, E., Dehouck, L., Sallusto, F., Gosselet, F., McGrath, J. L., Engelhardt, B., 2019. A silicon nanomembrane platform for the visualization of immune cell trafficking across the human blood-brain barrier under flow. J Cereb Blood Flow Metab 39 (3), 395-410.
Naegelen, I., Beaume, N., Plancon, S., Schenten, V., Tschirhart, E. J., Brechard, S., 2015. Regulation of Neutrophil Degranulation and Cytokine Secretion: A Novel Model Approach Based on Linear Fitting. J Immunol Res 2015, 817038.
Obermeier, B., Daneman, R., Ransohoff, R. M., 2013. Development, maintenance and disruption of the blood-brain barrier. Nat Med 19 (12), 1584-1596.
Ortega, M. A., Fernandez-Garibay, X., Castano, A. G., De Chiara, F., Hernandez-Albors, A., Balaguer-Trias, J., Ramon-Azcon, J., 2019. Muscle-on-a-chip with an on-site multiplexed biosensing system for in situ monitoring of secreted IL-6 and TNF-alpha. Lab Chip 19 (15), 2568-2580.
Salminen, A. T., Tithof, J., Izhiman, Y., Masters, E. A., McCloskey, M. C., Gaborski, T. R., Kelley, D. H., Pietropaoli, A. P., Waugh, R. E., McGrath, J. L., 2020. Endothelial cell apicobasal polarity coordinates distinct responses to luminally versus abluminally delivered TNF-alpha in a microvascular mimetic. Integr Biol (Camb) 12 (11), 275-289.
Schilling, M., Strecker, J. K., Ringelstein, E. B., Kiefer, R., Schabitz, W. R., 2009. Turn-over of meningeal and perivascular macrophages in the brain of MCP-1-, CCR-2- or double knockout mice. Exp Neurol 219 (2), 583-585.
Song, Y., Sandford, E., Tian, Y., Yin, Q., Kozminski, A. G., Su, S. H., Cai, T., Ye, Y., Chung, M. T., Lindstrom, R., Goicochea, A., Barabas, J., Olesnavich, M., Rozwadowski, M., Li, Y., Alam, H. B., Singer, B. H., Ghosh, M., Choi, S. W., Tewari, M., Kurabayashi, K., 2020. Rapid single-molecule digital detection of protein biomarkers for continuous monitoring of systemic immune disorders. Blood.
Song, Y., Ye, Y., Su, S.-H., Stephens, A., Cai, T., Chung, M.-T., Han, M. K., Newstead, M. W., Yessayan, L., Frame, D., Humes, H. D., Singer, B. H., Kurabayashi, K., 2021a. A digital protein microarray for COVID-19 cytokine storm monitoring. Lab on a Chip.
Song, Y., Zhao, J., Cai, T., Stephens, A., Su, S.-H., Sandford, E., Flora, C., Singer, B. H., Ghosh, M., Choi, S. W., Tewari, M., Kurabayashi, K., 2021b. Machine Learning-Based Cytokine Microarray Digital Immunoassay Analysis. Biosensors and Bioelectronics, 113088.
Stowe, A. M., Wacker, B. K., Cravens, P. D., Perfater, J. L., Li, M. K., Hu, R. L., Freie, A. B., Stuve, O., Gidday, J. M., 2012. CCL2 upregulation triggers hypoxic preconditioning-induced protection from stroke. Journal of Neuroinflammation 9.
Su, S. H., Song, Y., Newstead, M. W., Cai, T., Wu, M., Stephens, A., Singer, B. H., Kurabayashi, K., 2021. Ultrasensitive Multiparameter Phenotyping of Rare Cells Using an Integrated Digital-Molecular-Counting Microfluidic Well Plate. Small 17 (31), e2101743. Williams, J. L., Holman, D. W., Klein, R. S., 2014. Chemokines in the balance: maintenance of homeostasis and protection at CNS barriers. Front Cell Neurosci 8, 154.
Wolff, A., Antfolk, M., Brodin, B., Tenje, M., 2015. In Vitro Blood-Brain Barrier Models—An Overview of Established Models and New Microfluidic Approaches. J Pharm Sci 104 (9), 2727-2746.
Yu, F., Kumar, N. D. S., Foo, L. C., Ng, S. H., Hunziker, W., Choudhury, D., 2020. A pump-free tricellular blood-brain barrier on-a-chip model to understand barrier property and evaluate drug response. Biotechnol Bioeng 117 (4), 1127-1136.
Zhang, Y., Noji, H., 2017. Digital Bioassays: Theory, Applications, and Perspectives (vol 89, pg 92, 2017). Analytical Chemistry 89 (24), 13675-13675.
Zhang, et al. 2017. Multisensor-integrated organs-on-chips platform for automated and continual in situ monitoring of organoid behaviors. Proc Natl Acad Sci USA 114 (12), E2293-E2302.
Zhu, J., He, J., Verano, M., Brimmo, A. T., Glia, A., Qasaimeh, M. A., Chen, P., Aleman, J. O., Chen, W., 2018. An integrated adipose-tissue-on-chip nanoplasmonic biosensing platform for investigating obesity-associated inflammation. Lab on a Chip 18 (23), 3550-3560.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A system comprising:
a) a first component comprising:
i) a housing block having a top surface, a bottom surface, and top reservoir recessed in said top surface,
wherein said top reservoir comprises: one or more side walls and a bottom wall in proximity to said bottom surface of said housing block and having a reservoir hole therein,
ii) a membrane chip comprising a membrane housing and a nanoporous membrane, wherein said membrane chip is configured to sit on or near said bottom wall of said top reservoir such that said nanoporous membrane protrudes through said reservoir hole out past said bottom surface of said housing block, and
iii) optionally an adhesive layer that is sized to stick to said bottom surface of said housing block, and
iv) a film layer with a top surface and a bottom surface, wherein said film layer has an opening therein, wherein said opening comprises a film perimeter wall that forms a bottom reservoir when: A) said top surface of said film layer is mated with said bottom surface of said housing block, optionally via said adhesive layer, and B) said bottom surface of said film layer is mated with a second component, optionally via pressure.

2. The system of claim 1, wherein said top reservoir and said bottom reservoir are fluidically connected through, and only through, said nanoporous membrane when said housing block, membrane chip, and said film are assembled together, optionally with said adhesive layer.

3. The system of claim 2, wherein said housing block, membrane chip, and said film are assembled together, optionally via said adhesive layer.

4. The system of claim 1, wherein said adhesive layer is present in said system and comprises two-sided film adhesive.

5. The system of claim 1, further comprising said second component.

6. The system of claim 5, wherein said second component comprises a microwell chip, wherein said microwell chip comprises a surface with a plurality of microwells therein.

7. The system of claim 6, wherein said plurality of microwells are in fluidic communication with said bottom reservoir when said first component is assembled and mated with said second component.

8. The system of claim 6, wherein said microwell chip comprises glass.

9. The system of claim 6, wherein said plurality of microwells comprises at least 1000 microwells.

10. The system of claim 6, wherein said plurality of microwells are present in at least two or at least three distinct arrays.

11. The system of claim 6, wherein each of said plurality of microwells comprises an analyte binding agent.

12. The system of claim 11, wherein said analyte comprises a cytokine or chemokine.

13. The system of claim 10, wherein each of said at least two or at least three distinct arrays comprises a different analyte binding agent configured to bind a different analyte.

14. The system of claim 1, wherein said top reservoir comprises a first suspension of cells, and optionally a stimulating agent or candidate therapeutic, wherein said stimulating agent is optionally LPS, and wherein said candidate therapeutic is optionally a small molecule, antibody or antigen binding fragment thereof, a nanoparticle, or a liposome.

15. The system of claim 14, wherein said first suspension of cells comprises endothelial cells, which has optionally formed a layer on said nanoporous membrane.

16. The system of claim 15, wherein said endothelial cells comprise brain microvascular endothelial cells.

17. The system of claim 5, wherein said first component is assembled and mated with said second component such that said bottom reservoir is formed, and wherein said bottom reservoir comprises a second suspension of cells.

18. The system of claim 17, wherein said second suspension of cells comprises neurological cells or brain cells.

19. The system of claim 18, wherein said neurological cells comprise neurons.

20. A device comprising: a first component which comprises:
a) a housing block having a top surface, a bottom surface, and top reservoir recessed in said top surface, wherein said top reservoir comprises: one or more side walls and a bottom wall in proximity to said bottom surface of said housing block and having a reservoir hole therein,
b) a membrane chip comprising a membrane housing and a nanoporous membrane, wherein said membrane chip is seated on or near said bottom wall of said top reservoir such that said nanoporous membrane protrudes through said reservoir hole out past said bottom surface of said housing block, and
c) optionally an adhesive layer that is stuck to said bottom surface of said housing block, and
d) a film layer with a top surface and a bottom surface, wherein said film layer has an opening therein, wherein said opening comprises a film perimeter wall that forms a bottom reservoir when: A) said top surface of said film layer is mated with said bottom surface of said housing block, optionally via said adhesive layer, and B) said bottom surface of said film layer is mated with a second component, optionally via pressure.

21. The device of claim 20, wherein said top reservoir and said bottom reservoir are fluidically connected through, and only through, said nanoporous membrane.

22. The device of claim 20, wherein said adhesive layer is present and comprises two-sided film adhesive.

23. A method comprising:
a) sealing said film layer of said first component of said device of claim 20 against a first location on a second component such that said bottom reservoir is formed,
wherein said second component comprises a microwell chip, wherein said first location on said microwell chip comprises a surface with a first plurality of microwells therein,
wherein each of said first plurality of microwells comprises analyte binding agents, and
wherein said sealing is such that said plurality of microwells are in fluidic communication with said bottom reservoir,
b) adding a first cell suspension comprising a plurality of cells to said top reservoir of said first component,
c) adding a stimulating agent and/or candidate therapeutic to said top reservoir of said first component under conditions such that at least some of said plurality of cells secrete one or more analytes that: i) pass through said nanoporous membrane, ii) flow into said bottom reservoir, and iii) flow into at least a portion of said first plurality of microwells; and
d) detecting any bound analytes in said a least a portion of said first plurality of microwells at a first time.

24. The method of claim 23, wherein said detecting is performed by optical detection of an added label.

25. The method of claim 23, further comprising: e) moving said first component to a second location on said second component such that said bottom reservoir remains formed, wherein said second location on said microwell chip comprises a surface with a second plurality of microwells therein, wherein said second plurality of microwells comprises analyte binding agents.

26. The method of claim 25, wherein said moving comprises sliding said first component across said second component from said first location to said second location.

27. The method of claim 23, wherein said top reservoir and said bottom reservoir are fluidically connected through, and only through, said nanoporous membrane.

28. The method of claim 23, wherein said adhesive layer is present in said system and comprises two-sided film adhesive.

29. The method of claim 23, wherein said microwell chip comprises glass.

30. The method of claim 23, wherein said first plurality of microwells comprises at least 1000 microwells.

31. The method of claim 23, wherein said first plurality of microwells are present in at least two or at least three distinct arrays.

32. The method of claim 23, wherein said analyte comprises a cytokine or chemokine.

33. The method of claim 23, wherein said first cell suspension comprises endothelial cells, which have optionally formed a layer on said nanoporous membrane.

34. The method of claim 33, wherein said endothelial cells comprise brain microvascular endothelial cells.

35. The method of claim 23, wherein said bottom reservoir comprises a second suspension of cells comprises neurological cells or brain cells.

36. The method of claim 35, wherein said neurological cells comprise neurons.

37. A method comprising;
a) adding a first cell suspension comprising a plurality of cells to said top reservoir of said first component of said device of claim 20,
wherein said first component is sealed against a second component such that said bottom reservoir is formed,
wherein said second component comprises a microwell chip, wherein said first location on said microwell chip comprises a surface with a first plurality of microwells therein,
wherein each of said first plurality of microwells comprises analyte binding agents, and
wherein said sealing is such that said plurality of microwells are in fluidic communication with said bottom reservoir,
b) adding a stimulating agent and/or candidate therapeutic to said top reservoir of said first component under conditions such that at least some of said plurality of cells secrete one or more analytes that: i) pass through said nanoporous membrane, ii) flow into said bottom reservoir, and iii) flow into at least a portion of said first plurality of microwells; and
c) detecting any bound analytes in said at least a portion of said first plurality of microwells at a first time.
